(19) United States
(12) Patent Application Publication
(10) Pub. No.: US 2019/0153379 A1
(43) Pub. Date: May 23, 2019

(54) CELL CULTURE INCUBATOR AND METHOD FOR OPERATING SAME

(71) Applicant: CHINA INNOVATION INSTRUMENT CO., LTD, Ningbo (CN)

(72) Inventors: Luhong WEN, Ningbo (CN); Huanhuan HONG, Ningbo (CN); Zhenzhi SHI, Ningbo (CN); Lulu SHI, Ningbo (CN); Jianqin GAN, Ningbo (CN)

(21) Appl. No.: 16/257,341

(22) Filed: Jan. 25, 2019

Related U.S. Application Data
(63) Continuation of application No. PCT/CN2017/109953, filed on Nov. 8, 2017.

(30) Foreign Application Priority Data
Nov. 10, 2016 (CN) 201610988322.0

(51) Int. Cl. C12M 1/00

(52) U.S. Cl. CPC C12M 41/14; C12M 23/34

(57) ABSTRACT

A cell culture incubator includes a first incubator body; a second incubator body arranged inside the first incubator body, a closed space being formed between the second incubator body and the first incubator body; a first incubator door arranged on a front side of the first incubator body, a sealing member being arranged between the first incubator body and the first incubator door; a temperature measuring device arranged inside the second incubator body; a heating device arranged on an inner side of the first incubator door and a side of the second incubator body; a fan heater arranged in the closed space; and an insulation device arranged on an outer side or an inner side of the first incubator body.

### CROSS-REFERENCE TO RELATED APPLICATIONS

This application is a continuation of International Patent Application No. PCT/CN2017/109953, filed on Nov. 8, 2017, which claims the benefit of priority from Chinese Application No. 201610988322.0, filed on Nov. 10, 2016. The contents of the aforementioned applications, including any intervening amendments thereto, are incorporated herein by reference.

### TECHNICAL FIELD

The present disclosure relates to cell culture, and in particular to a cell culture incubator and a method for operating the same.

### BACKGROUND

In the fields of medicine, life and health, environmental science, etc., a cell culture workstation provides an ISO 5- or GMP-grade culture environments in an ultraclean closed space. Incubator with built-in features is a device for culturing cells under the simulated in-vivo physiological environment. The incubator is capable of controlling temperature with characteristics such as constant temperature, high temperature uniformity, rapid temperature recovery, and no condensation of water caused by temperature difference. Existing incubators include the following heating modes.

1. Water-Jacketed Incubator

The stability of this heating mode depends largely on the stability and response time of the heating wire; that is, the heating wire with a reasonable thickness and diameter is selected to reduce the internal consumption of the heating wire. Further, the water tank requires water feed sometimes, and it is necessary to clean, empty and monitor the water tank from time to time, which is particularly difficult for incubator design.

2. Air-Jacketed Incubator

This heating mode has led to heating uniformity. For an internal circulation incubator, the air ducts and air vents additionally provided on the inner wall of the internal circulation cavity result in high design requirements and high cost. While, the external circulation incubator with a simple structure has non-uniform thermal conduction.

3. Direct-Heated Incubator

This heating mode has a high-precision temperature control. Such six-sided heating mode can essentially eliminate the condensation of water on the glass door. However, it may cause non-uniform temperature field distribution in the incubator body, resulting in the inconsistency of the cell culture environment. Moreover, when a failure occurs on one or more sides of the inner wall of the incubator, the stability of the temperature control in the chamber is greatly affected. Particularly, the replacement of heating elements and other components will bring great difficulty in creating a stable and continuous cell culture environment.

### SUMMARY

In order to solve the above deficiencies, the present disclosure provides a cell culture incubator with an accurate temperature control and a good temperature uniformity.

The object of the present disclosure is realized by the following technical solutions.

The cell culture incubator includes:

a first incubator body;

a second incubator body arranged inside the first incubator body, a closed space being formed between the second incubator body and the first incubator body;

a first incubator door arranged on a front side of the first incubator body, a sealing member being arranged between the first incubator body and the first incubator door;

a temperature measuring device arranged inside the second incubator body;

a plurality of heating devices arranged on an inner side of the first incubator door and on a side of the second incubator body;

a fan heater arranged in the closed space; and

an insulation device arranged on an outer side or an inner side of the first incubator body.

Another object of the present disclosure is to provide a cell culture workstation with precise temperature control and good temperature uniformity.

The cell culture workstation of the present invention includes a cabinet, a bench and glove ports arranged on a front side of the cabinet. The cell culture workstation further includes the cell culture incubator as described above.

The front side of the first incubator body is fixed to a rear side of the cabinet. The rear side of the cabinet is provided with a through hole corresponding to position and size of a first incubator door.

Another object of the present disclosure is to provide a method for operating the cell culture incubator.

The method for operating the cell culture incubator of the present invention includes the following steps:

(1) closing the first incubator door, and turning on the fan heater and the heating devices until a temperature measured by the temperature measuring device reaches a set value; and

(2) turning off the fan heater and then turning off the heating devices.

The present invention has the following beneficial effects compared to the prior art:

1. The culture incubator has the advantages of higher accuracy and precision of temperature control, good temperature field distribution uniformity and good consistency of culture environment;

2. Combination of the air-jacketed constant-temperature heating mode and the wrapped direct-heated manner effectively solves the problem of condensation of water on the transparent incubator doors and in the air ducts. Also, this leads to high temperature control efficiency and rapid temperature recovery.

3. The structure of the culture incubator is simple and compact.

### BRIEF DESCRIPTION OF THE DRAWINGS

The present invention will become more apparent with reference to the accompanying drawings. It should be understood by those skilled in the art that the drawings are merely illustrative of the present invention without limiting the protection scope of the present disclosure. In the drawings:

FIG. 1 is a schematic diagram of the structure of a cell culture incubator according to a first embodiment of the present invention.

FIG. 2 is a sectional view taken along A-A of the cell culture incubator according to the first embodiment of the present invention.

FIG. 3 is a schematic diagram of the structure of a cell culture workstation according to a second embodiment of the present invention.

### DETAILED DESCRIPTION OF EMBODIMENTS

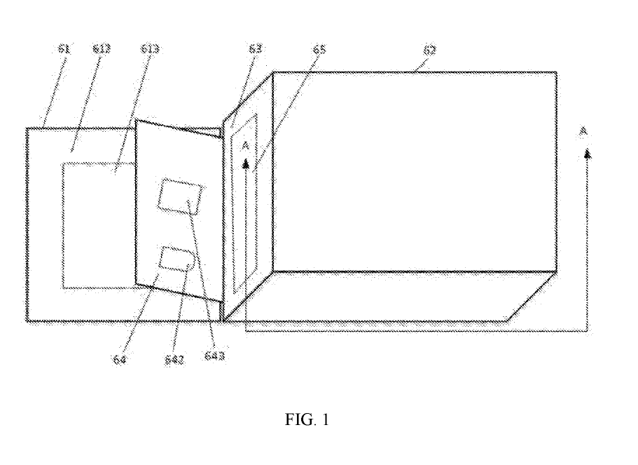

FIGS. 1-3 and the following descriptions illustrate optional embodiments of the present invention to teach a person skilled in the art how to implement and reproduce the present invention. Some conventional aspects have been simplified or omitted so make the present invention more apparent. It should be understood by those skilled in the art that variants or substitutions derived from these implementations shall fall into the scope of the present disclosure. It should be understood by those skilled in the art that the following features can be combined in various ways to form a plurality of variants of the present disclosure. Therefore, the present disclosure is not limited to the following optional implementations, and instead, shall be defined by the appended claims and equivalents thereof.

### EXAMPLE 1

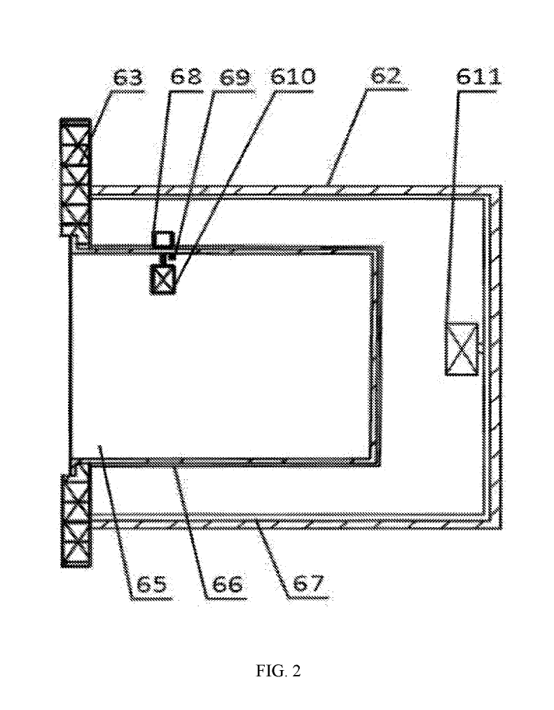

FIGS. 1-2 schematically show the structure of a cell culture incubator according to the present embodiment. As shown in FIGS. 1-2, the cell culture incubator includes:

a first incubator body 62 made of a polished electrolytic stainless steel material;

a second incubator body 65 arranged inside the first incubator body, a closed space being formed between the second incubator body and the first incubator body;

a first incubator door 61 arranged on a front side 63 of the first incubator body, a sealing member being arranged between the first incubator body and the first incubator door;

a second incubator door 64 arranged on the front side of the first incubator body and on an inner side of the first incubator door, the second incubator door being made of a transparent material, for example, glass; the second incubator door being provided with at least two through holes and transparent doors 642 and 643 configured to block the through holes;

a temperature measuring device 69, for example, a temperature sensor arranged inside the second incubator body;

a plurality of heating devices 66 arranged on an inner side of the first incubator door and on a side of the second incubator body; particularly, six heating elements arranged on the inner side of the first incubator door and on an upper side, a lower side, a rear side, a right side and a left side of the second incubator body, respectively;

a fan heater 611 arranged in the closed space, for example, on an inner wall, opposite to the rear side of the second incubator body, of the first incubator body;

an insulation device 67 arranged on an outer side or an inner side of the first incubator body; for example, on an inner wall of the first incubator body or on an outer wall of the second incubator body, such that an insulated closed space is formed between the first incubator body and the second incubator body;

a fan 610 arranged in a space formed by the second incubator body; and

a motor 68 that controls a direction of rotation of the fan allowing for a uniformity gas distribution in the second incubator body by means of appropriate wind direction.

A method for operating the cell culture incubator according to this embodiment includes the following steps:

(1) closing the first incubator door and the second incubator door so that an closed space is formed inside the second incubator body; turning on the heating elements and running the fan 610, the fan heater 611 at the same time; when the temperature value measured by two temperature sensors 69 in the second incubator body 65 reaches 35° C., controlling the heating power to slowly raise the temperature to 37° C.; and

(2) turning off the fan heater 611 and then turning off the fan 610 and the six heating elements.

When the internal space of the second incubator body 65 is heated unequally, the output power of the heating elements is adjusted by controlling the corresponding wall is heated or the heating power. Meanwhile, the motor 68 is controlled enabling the auto-rotation of the fan 610 for the control of wind direction, which functions to finely adjust the temperature uniformity in the second incubator body 65. In an extreme case where failures of the heating elements occur, in order to ensure the survival rate of the cultured cells, the output power of the workable heating elements is reduced to slow down the temperature reduction in the second incubator body 65. The fan 610 is then controlled to turn at a large angle by adjusting the motor 68, and the output power of the fan heater 611 is increased at the same time for stable temperature control. In this way, it is ensured that the cells already placed in the second incubator body 65 is cultured continuously and normally until the end of culture.

EXAMPLE 2

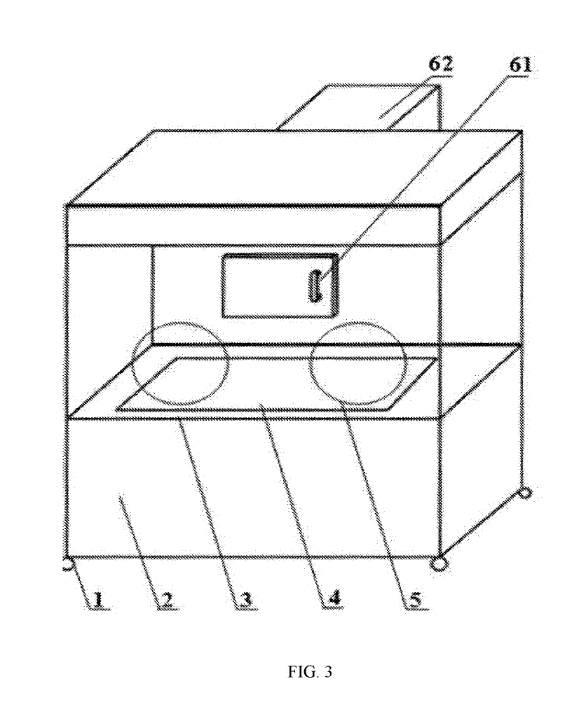

FIG. 3 schematically shows a structure of a cell culture workstation according to this embodiment of the present invention.

As shown in FIG. 3, the cell culture workstation includes a cabinet 2, a bench 4, glove ports 5 arranged on a front side of the cabinet 2, a transparent plate 3, a movable wheel 1, and the cell culture incubator of Example 1. The front side of the first incubator body 62 is fixed to the rear side of the cabinet. The rear side of the cabinet is provided with a through hole corresponding to position and size of the first incubator door 61.

What is claimed is:

1. A cell culture incubator, comprising:
a first incubator body;
a second incubator body arranged inside the first incubator body, a closed space being formed between the second incubator body and the first incubator body;
a first incubator door arranged on a front side of the first incubator body, a sealing member being arranged between the first incubator body and the first incubator door;
a temperature measuring device arranged inside the second incubator body;
a plurality of heating devices arranged on an inner side of the first incubator door and on a side of the second incubator body;
a fan heater arranged in the closed space; and
an insulation device arranged on an outer side or an inner side of the first incubator body.

2. The cell culture incubator according to claim 1, wherein the heating device is arranged on an outer wall of the second incubator body, and the insulation device is arranged on an inner wall of the first incubator body.

3. The cell culture incubator according to claim 1, wherein the fan heater is arranged on an inner wall of a rear side of the first incubator body that is opposite to a rear side of the second incubator body.

4. The cell culture incubator according to claim 1, further comprising:
a fan arranged in a space formed by the second incubator body.

5. The cell culture incubator according to claim 4, further comprising:
a motor for controlling a direction of rotation of the fan.

6. The cell culture incubator according to claim 1, further comprising:
a second incubator door arranged on the front side of the first incubator body and the inner side of the first incubator door; wherein the second incubator door is made of a transparent material.

7. The cell culture incubator according to claim 6, wherein the second incubator door is provided with at least two through holes and transparent doors for blocking the through holes.

8. A cell culture workstation, comprising:
a cabinet,
a bench, and
glove ports arranged on a front side of the cabinet, and
a cell culture incubator of claim 1;
wherein the front side of the first incubator body is fixed to a rear side of the cabinet; the rear side of the cabinet is provided with a through hole corresponding to position and size of the first incubator door.

9. A method for operating the cell culture incubator of claim 1, comprising:
(1) closing the first incubator door, and turning on the fan heater and the heating devices until a temperature measured by the temperature measuring device reaches a set value; and
(2) turning off the fan heater and then turning off the heating devices.
